(19) United States
(12) Patent Application Publication
(10) Pub. No.: US 2009/0053229 A1
(43) Pub. Date: Feb. 26, 2009

(54) Methods of Treating Conditions Involving Neuronal Degeneration

(76) Inventors: Daniel H.S. Lee, Sudbury, MA (US); Dinah W.Y. Sah, Boston, MA (US); Kwok Fai So, Hong Kong (CN); Wutian Wu, Hong Kong (CN)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 11/914,195

(22) PCT Filed: May 12, 2006
(86) PCT No.: PCT/US2006/018484

§ 371 Date: May 9, 2008

Related U.S. Application Data
(60) Provisional application No. 60/679,995, filed on May 12, 2005.
(60) Provisional application No. 60/735,187, filed on Nov. 10, 2005.

(51) Int. Cl. A61K 39/395; A61K 38/17; A61P 27/16; A61P 27/02

(52) U.S. Cl. 424/139.1; 514/12; 424/178.1

(57) ABSTRACT

The invention provides methods for treating conditions of the eye involving death or degeneration of retinal ganglion cells, including glaucoma, by the administration of Nogo receptor-1 antagonists.

### BACKGROUND OF THE INVENTION

1. Field of the Invention

This invention relates to neurobiology and pharmacology. More particularly, it relates to methods of treating conditions involving neuronal degeneration by the administration of Nogo receptor-1 antagonists, including degeneration of sensory neurons such as retinal ganglion cells and hairy cells.

2. Background Art

Optical neuropathies are a group of eye diseases encompassing various clinical presentations and etiologies. Glaucoma is an exemplary optical neuropathy which includes pathological changes in the optic nerve, visible on the optic disk, and corresponding visual field loss, resulting in blindness if untreated. Glaucoma also is associated with increased intraocular pressure, but other factors are involved.

Current therapies for glaucoma are directed at decreasing intraocular pressure. Medical therapy includes topical ophthalmic drops or oral medications that reduce the production or increase the outflow of intraocular fluid. However, these drug therapies for glaucoma are sometimes associated with significant side effects, such as headache, blurred vision, allergic reactions, death from cardiopulmonary complications, and potential interactions with other drugs. Surgical therapies also are used, but they also have numerous disadvantages and modest success rates.

Accordingly, there remains a need for additional treatment methods for optical neuropathies, including glaucoma and other conditions characterized by degeneration or death of retinal ganglion cells (RGCs).

### BRIEF SUMMARY OF THE INVENTION

The invention relates to methods of treating conditions involving degeneration or death of sensory neurons. For example, the invention relates to methods of treating optical neuropathies such as glaucoma and other conditions characterized by degeneration or death of RGCs. The methods of the invention comprise the administration of agents which interfere with Nogo receptor (NgR)-mediated neuronal growth inhibition such as, e.g., Nogo receptor-1 antagonists.

In some embodiments, the invention provides a method of promoting regeneration or survival of retinal ganglion cells (RGCs) in a mammal displaying signs or symptoms of a condition involving RGC death, comprising administering to the mammal a therapeutically effective amount of an NGR1 antagonist. In some embodiments, the NGR1 antagonist is administered directly into the eye. In some embodiments, the NGR1 antagonist is administered intravitreally. In some embodiments, the NgR1 antagonist is administered via a capsule implant. In some embodiments, the mammal suffers from one or more optical neuropathies, e.g., glaucoma.

The inhibition of neuronal growth mediated by NgR (and associated factors), and therapeutic approaches involving the manipulation of the NgR signaling pathway, are generally described in, e.g., Lee et al., Nature Reviews 2:1-7 (2003).

In some embodiments, the methods of the invention use an NgR1 antagonist that comprises a soluble form of a mammalian NGR1. In some embodiments, the soluble form of a mammalian NgR1 comprises amino acids 26 to 310 of SEQ ID NO: 3 with up to ten conservative amino acid substitutions. In some embodiments, the soluble form of mammalian NgR1 comprises amino acids 26 to 344 of SEQ ID NO: 4 with up to ten conservative amino acid substitutions. In some embodiments, the soluble form of mammalian NGR1 comprises amino acids 27 to 310 of SEQ ID NO: 5 with up to ten conservative amino acid substitutions. In some embodiments, the soluble form of mammalian NGR1 comprises amino acids 27 to 344 of SEQ ID NO: 6 with up to ten conservative amino acid substitutions. In some embodiments, the soluble form of mammalian NGR1 lacks a functional signal peptide.

In some embodiments, the soluble form of mammalian NgR1 comprises amino acids 26-310 of SEQ ID NO:3 except that at least one cysteine residue is substituted with a different amino acid. In some embodiments, the soluble form of mammalian NgR1 comprises amino acids 27-310 of SEQ ID NO:5 except that at least one cysteine residue is substituted with a different amino acid. In some embodiments, C266 is substituted with a different amino acid. In some embodiments, C309 is substituted with a different amino acid. In some embodiments, both C266 and C309 are substituted with different amino acids. In some embodiments, the different amino acid is alan me.

In some embodiments, the soluble form of a mammalian NgR1 further comprises a fusion moiety. In some embodiments, the fusion moiety is an immunoglobulin moiety. In some embodiments, the immunoglobulin moiety is an Fc moiety.

In some embodiments, the NGR1 antagonist comprises an antibody or antigen-binding fragment thereof that binds to a mammalian NgR1. In some embodiments, the antibody is selected from the group consisting of a polyclonal antibody, a monoclonal antibody, a Fab fragment, a Fab′ fragment, a F(ab′)2 fragment, an Fv fragment, an Fd fragment, a diabody, and a single-chain antibody. In some embodiments, the antibody or antigen-binding fragment thereof binds to an polypeptide bound by a monoclonal antibody produced by a hybridoma selected from the group consisting of: HB 7E11 (ATCC® accession No. PTA-4587), HB 1H2 (ATCC® accession No. PTA-4584), HB 3G5 (ATCC® accession No. PTA-4586), HB 5B10 (ATCC® accession No. PTA-4588) and HB 2F7 (ATCC® accession No. PTA-4585). In some embodiments, the polypeptide comprises an amino acid sequence selected from the group consisting of: AAAFGLTLLEQLDLSDNAQLR (SEQ ID NO: 7); LDLSDNAQLR (SEQ ID NO: 8); LDLSDDAELR (SEQ ID NO: 9); LDLASDNAQLR (SEQ ID NO: 10); LDLASDDAELR (SEQ ID NO: 11); LDALSDNAQLR (SEQ ID NO: 12); LDALSDDAELR (SEQ ID NO: 13); LDLSSDNAQLR (SEQ ID NO: 14); LDLSSDEAELR (SEQ ID NO: 15); DNAQLRVVDPTT (SEQ ID NO: 16); DNAQLR (SEQ ID NO: 17); ADLSDNAQLRVVDPTT (SEQ ID NO: 18); LALSDNAQLRVVDPTT (SEQ ID NO: 19); LDLSDNAALRVVDPTT (SEQ ID NO: 20); LDLSDNAQLHVVDPTT (SEQ ID NO: 21); and LDLSDNAQLAVVDPTT (SEQ ID NO: 22). In one embodiment, the NgR1 antagonist is monoclonal antibody 1D9.

In some embodiments, the therapeutically effective amount is from 0.001 mg/kg to 10 mg/kg. In some embodiments, the therapeutically effective amount is from 0.01 mg/kg to 1.0 mg/kg. In some embodiments, the therapeutically effective amount is from 0.05 mg/kg to 0.5 mg/kg.

In some embodiments, the invention provides a method of treating an optical neuropathy in a mammal, comprising administering to the mammal a therapeutically effective amount of an NGR1 antagonist. In certain embodiments, the optical neuropathy is glaucoma.

### BRIEF DESCRIPTION OF THE DRAWINGS/FIGURES

FIG. 1A-1B show that NGR1 is expressed in rat retinal ganglion cells (RGCs). GCL, ganglion cell layer; INL, innemuclear layer; ONL, outernuclearlayer. Scale bars: A, 25 μm; B, 200 μm; C, 25 μm.

FIG. 2 shows a model of the binding of the anti-rNgR1 antibody, 1D9, to the soluble fragment of rNgR1 (srNgR310).

FIG. 3 shows the dose-response effect of Nogo receptor-1 antagonist (srNgR310-Fc) treatment on survival of dorsal root ganglion (DRG) neurons in vitro.

FIGS. 4A-4C shows the optic nerve transection model. Optic nerve was transected at 1.5 mm from optic disc. Right eyes are the experimental eyes. FG=fluorogold

FIG. 5 shows the effect of sNgR1(27-310)-Fc protein on survival of injured retinal ganglion cells (RGCs) after optic nerve transection. sNgR1(27-310)-Fc protein treatment promotes the survival of injured RGCs after optic nerve transection. P-value represents comparison to the other groups.

FIG. 6 shows the glaucoma model. Elevated intraocular pressure is induced in the right eye using Argon laser photocoagulation at the limbal and three episcleral veins.

FIGS. 7A-7C shows the effect of Nogo receptor-1 antagonist (srNgR310-Fc) treatment on survival of RGCs in vivo in a rat glaucoma model. FIGS. 7A and 7B. sNgR1(27-310)-Fc treatment promotes the survival of injured RGCs after induction of ocular hypertension. FIG. 7C. Treatment with sNgR1(27-310)-Fc had no effect in lowering the intraocular pressure (IOP) after laser treatment.

FIG. 8 shows the effect of 1D9 treatment on survival of DRG neurons in vitro. 1D9 treatment protected serum deprived rat p2 DRG neurons for 6 days in culture. Mean values are presented and error bars indicate S.E.M.

FIGS. 9A-9C show the effect of a Fab fragment of a monoclonal anti-NgR1 antibody (1D9) on survival of DRG neurons in the optic nerve transection model. FIG. 9A. Bar chart representing the mean percentage of survival of RGC's with rat 1D9 (070) and control treatment after transection of the optic nerve. FIG. 9B. Bar chart representing the mean percentage of survival of RGC's with rat 1D9 (052) and control treatment after transection of the optic nerve. FIG. 9C. Bar chart representing the mean percentage of loss of RGC's with rat 1D9 (070) and control treatment after transection of the optic nerve, the asterisks indicate statistical significance (P<0.001) compared to the PBS group. Error bars indicate S.E.M.

FIGS. 10A and 10B show the effect of 1D9 on survival of RGCs ill vivo in a rat glaucoma model. FIG. 10A. 1D9 treatment promotes the survival of injured RGCs after induction of ocular hypertension compared to the PBS group. P<0.01, compared to PBS group. FIG. 10B. Ocular IOP measurement in control (PBS) and treatment (1D9) groups. Intravitreal injection of 1D9 had no effect in lowering the IOP after laser treatment. All animals received two laser photocoagulations. An increase of about 1.7 times in IOP was observed in the laser-treated eyes in all groups.

FIG. 11 shows the effect of Nogo receptor-1 antagonist (Ala-Ala-rNgR310-Fc and Ala-Ala-hNgR310-Fc) treatment on survival of RGCs in vivo in a rat glaucoma model. Both Ala-Ala-rNgR310-Fc and Ala-Ala-hNgR310-Fc treatment promote the survival of injured RGCs after induction of ocular hypertension.

### DETAILED DESCRIPTION OF THE INVENTION

Unless defined otherwise, all technical and scientific terms used herein have the same meaning as commonly understood by one of ordinary skill in the art to which this invention belongs. In case of conflict, the present application including the definitions will control. Also, unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present invention, suitable methods and materials are described below. The materials, methods and examples are illustrative only and are not intended to be limiting. Other features and advantages of the invention will be apparent from the detailed description and from the claims.

Throughout this specification and claims, the word “comprise,” or variations such as “comprises” or “comprising,” indicate the inclusion of any recited integer or group of integers but not the exclusion of any other integer or group of integers.

In order to further define this invention, the following terms and definitions are provided. As used herein, “antibody” means an intact immunoglobulin, or an antigen-binding fragment thereof. Antibodies of this invention can be of any isotype or class (e.g., M, D, G, E and A) or any subclass (e.g., G1-4, A1-2) and can have either a kappa (κ) or lambda (λ) light chain.

As used herein, “humanized antibody” means an antibody in which at least a portion of the non-human sequences are replaced with human sequences. Examples of how to make humanized antibodies may be found in U.S. Pat. Nos. 6,054,297, 5,886,152 and 5,877,293.

As used herein, a “therapeutically effective amount” refers to an amount effective, at dosages and for periods of time necessary, to achieve the desired therapeutic result.

As used herein, a “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

As used herein, a “patient” means a mammal, e.g., a human.

As used herein, “fusion protein” means a protein comprising a polypeptide fused to another, generally heterologous, polypeptide.

As used herein, a “Nogo receptor antagonist” means any molecule that inhibits the binding of Nogo receptor-1 to a ligand (e.g., NogoA, NogoB, NogoC, MAG, OM-gp). Nogo receptor antagonists include, but are not limited to, synthetic or native-sequence peptides, small molecules and antibodies. Exemplary NgR antagonists are described in detail elsewhere herein.

As used herein, “Nogo receptor polypeptide” includes both full-length Nogo receptor-1 protein and fragments thereof.

### Nogo Receptor Antagonists

The present invention is based on the discovery that Nogo receptor antagonists may be used to treat conditions involving death or degeneration of RGCs, including glaucoma. Nogo receptor antagonists of the present invention promote regeneration or survival of sensory neurons. Certain Nogo receptor antagonists of the present invention promote regeneration or survival of sensory neurons, but do not promote neurite outgrowth of CNS neurons. Any Nogo receptor antagonist may be used in the methods of the invention. For example, Nogo receptor antagonists that may be used in the methods of the invention include, but are not limited to: soluble Nogo receptor polypeptides; antibodies to the Nogo receptor protein and antigen-binding fragments thereof; and small molecule antagonists. Nogo receptor antagonists also include antibodies and other compounds (including polypeptides and small molecules) that interact with and/or bind to Nogo receptor ligands, such as, e.g., NogoA, NogoB, NogoC, MAG, OM-gp. For example, the Nogo receptor antagonist may, in certain embodiments, be a MAG derivative (see, e.g., U.S. Patent Appl. No. 2004-012-1341) or an OM-gp-specific binding agent (see, e.g., U.S. Patent Appl. No. 2003-011-3326).

### Soluble Nogo Receptor-1 Polypeptides

In some embodiments of the invention, the antagonist is a soluble Nogo receptor-1 polypeptide (Nogo receptor-1 is also variously referred to as “Nogo receptor,” “NogoR,” “NogoR-1,” “NgR,” “NgR-1”, NgR1, and NGR1). Full-length Nogo receptor-1 consists of a signal sequence, a N-terminus region (NT), eight leucine rich repeats (LRR), a LRRCT region (a leucine rich repeat domain C-terminal of the eight leucine rich repeats), a C-terminus region (CT) and a GPI anchor. The sequences of full-length human and rat Nogo receptors are shown in Table 1.

TABLE 1

Sequences of Human and Rat Nogo receptor-1
Polypeptides

| | |
|---|---|
| Full-length | MKRASAGGSRLLAWVLWLQAWQVAAPCPGACVCYN |
| human | EPKVTTSCPQQGLQAVPVGIPAASQRIFLHGNRISHV |
| Nogo receptor | PAASFRACRNLTILWLHSNVLARIDAAAFTGLALLEQ |
| SEQ ID NO: 1 | LDLSDNAQLRSVDPATFHGLGRLHTLHLDRCGLQELG |
| | PGLFRGLAALQYLYLQDNALQALPDDTFRDLGNLTHL |
| | FLHGNRISSVPERAFRGLHSLDRLLLHQNRVAHVHPH |
| | AFRDLGRLMTLYLFANNLSALPTEALAPLRALQYLRL |
| | NDNPWVCDCRARPLWAWLQKFRGSSSEVPCSLPQRLA |
| | GRDLKRLAANDLQGCAVATGPYHPIWTGRATDEEPLG |
| | LPKCCQPDAADKA |
| Full-length | MKRASSGGSRLPTWVLWLQAWRVATPCPGACVCYNE |
| rat Nogo | PKVTTSRPQQGLQAVPAGIPASSQRIFLHGNRISYVP |
| receptor | AASFQSCRNLTILWLHSNALAGIDAAAFTGLTLLEQL |
| SEQ ID NO: 2 | DLSDNAQLRVVDPTTFRGLGHLHTLHLDRCGLQELGP |
| | GLFRGLAALQYLYLQDNNLQALPDNTFRDLGNLTHLF |
| | LHGNRIPSVPEHAFRGLHSLDRLLLHQNVARVHPHAF |
| | RDLGRLMTLYLFANNLSMLPAEVLVPLRSLQYLRLND |
| | NPWVCDCRARPLWAWLQKFRGSSSGVPSNLPQRLAGR |
| | DLKRLATSDLEGCAVASGPFRPFQTNQLTDEELLGLP |
| | KCCQPDAADKA |

Soluble Nogo receptor polypeptides used in the methods of the invention comprise an NT domain; 8 LRRs and an LRRCT domain and lack a signal sequence and a functional GPI anchor (i.e., no GPI anchor or a GPI anchor that fails to efficiently associate to a cell membrane). Suitable polypeptides include, for example, amino acids 26-310 (SEQ ID NO: 3) and 26-344 (SEQ ID NO: 4) of the human Nogo receptor and amino acids 27-310 (SEQ ID NO: 5) and 27-344 (SEQ ID NO: 6) of the rat Nogo receptor (Table 2). Additional polypeptides which may be used in the methods of the invention are described, for example, in International Patent Applications PCT/US02/32007 and PCT/US03/25004.

TABLE 2

Soluble Nogo receptor Polypeptides from Human
and Rat

| | |
|---|---|
| Human 26-310 | PCPGACVCYEPKVTTSCPQQGLQAVPVGIPSQRIF |
| SEQ ID NO: 3 | LHGNRISHVPAASFRACRNLTILWLHSNVLARIDAAA |
| | FTGLALLEQLDLSDNAQLRSVDPATFHGLGRLHTLHL |
| | DRCGLQELGPGLFRGLAALQYLYLQDNALQALPDDTF |
| | RDLGNLTHLFLHGNRISSVPERAFRGLHSLDRLLLHQ |
| | NRVAHVHPHAFRDLGRLMTLYLFANNLSALPTEALAP |
| | LRALQYLRLNDNPWVCDCRARPLWAWLQKFRGSSSEV |
| | PCSLPQRLAGRDLKRLAADLQGCA |
| Human 26-344 | PCPGACVCYNEPKVTTSCPQQGLQAVPVGIPAASQRI |
| SEQ ID NO: 4 | FLHGNRISHVPAASFRACRNLTILWLHSNVLARIDAA |
| | AFTGLALLEQLDLSDNAQLRSVDPATFHGLGRLHTLH |
| | LDRCGLQELGPGLFRGLAALQYLYLQDNALQALPDDT |
| | FRDLGNLTHLFLHGNRISSVPERAFRGLHSLDRLLLH |
| | QNRVAHVHPHAFRDLGRLMTLYLFANNLSALPTEALA |
| | PLRALQYLRLNDNPWVCDCRARPLWAWLQKFRGSSSE |
| | VPCSLPQRLAGRDLKRLAANDLQGCAVATGPYHPIWT |
| | GRATDEEPLGLPKCCQPDAADKA |
| Rat 27-310 | CPGACVCYNEPKVTTSRPQQGLQAVPAGIPASSQRIF |
| SEQ ID NO: 5 | LHGNRISYVPAASFQSCRNLTILWLHSNALAGIDAAA |
| | FTGLTLLEQLDLSDNAQLRVVDPTTFRGLGHLHTLHL |
| | DRCGLQELGPGLFRGLAALQYLYLQDNNLQALPDNTF |
| | RDLGNLTHLFLHGNRIPSVPEHAFRGLHSLDRLLLHQ |
| | NHVARVHPHAFRDLGRLMTLYLFANNLSMLPAEVLVP |
| | LRSLQYLRLNDNPWVCDCRARPLWAWLQKFRGSSSGV |
| | PSNLPQRLAGRDLKRLATSDLEGCA |
| Rat 27-344 | CPGACVCYNEPKVTTSRPQQGLQAVPAGIPASSQRIF |
| SEQ ID NO: 6 | LHGNRISYVPAASFQSCRNLTILWLHSNALAGIDAAA |
| | FTGLTLLEQLDLSDNAQLRVVDPTTFRGLGHLHTLHL |
| | DRCGLQELGPGLFRGLAALQYLYLQDNNLQALPDNTF |
| | RDLGNLTHLFLHGNRIPSVPEHAFRGLHSLDRLLLHQ |
| | NHVARVHPHAFRDLGRLMTLYLFANNLSMLPAEVLVP |
| | LRSLQYLRLNDNPWVCDCRARPLWAWLQKFRGSSSGV |
| | PSNLPQRLAGRDLKRLATSDLEGCAVASGPFRPFQTN |
| | QLTDEELLGLPKCCQPDAADKA |
| Human 26-310 | PCPGACVCYNEPKVTTSCPQQGLQAVPVGIPAASQRI |
| with Ala-Ala | FLHGNRISHVPAASFRACRNLTILWLHSNVLARIDAA |
| substitutions | AFTGLALLEQLDLSDNAQLRSVDPATFHGLGRLHTLH |
| at positions | LDRCGLQELGPGLFRGLAALQYLYLQDNALQALPDDT |
| 266 and 309 | FRDLGNLTHLFLHGNRISSVPERAFRGLHSLDRLLLH |
| SEQ ID NO:23 | QNRVAHVHPHAFRDLGRLMTLYLFANNLSALPTEALA |
| | PLRALQYLRLNDNPWVCDARARPLWAWLQKFRGSSSE |
| | VPCSLPQRLAGRDLKRLAANDLQGAA |
| Rat 27-310 | CPGACVCYNEPKVTTSRPQQGLQAVPAGIPASSQRIF |
| with Ala-Ala | LHGNRISYVPAASFQSCRNLTILWLHSNALAGIDAAA |
| substitutions | FTGLTLLEQLDLSDNAQLRVVDPTTFRGLGHLHTLHL |
| at positions | DRCGLQELGPGLFRGLAALQYLYLQDNNLQALPDNTF |
| 266 and 309 | RDLGNLTHLFLHGNRIPSVPEHAFRGLHSLDRLLLHQ |
| SEQ ID NO:24 | NHVARVHPHAFRDLGRLMTLYLFANNLSMLPAEVLVP |
| | LRSLQYLRLNDNPWVCDARARPLWAWLQKFRGSSSGV |
| | PSNLPQRLAGRDLKRLATSDLEGAA |

A soluble Nogo receptor polypeptide that is a component of a fusion protein also may be used in the methods of the invention. In some embodiments, the heterologous moiety of the fusion protein is an immunoglobulin constant domain. In some embodiments, the immunoglobulin constant domain is a heavy chain constant domain. In some embodiments, the heterologous polypeptide is an Fc fragment. In some embodiments, the Fc is joined to the C-terminal end of a soluble Nogo receptor polypeptide. In some embodiments, the fusion Nogo receptor protein is a dimer.

An exemplary soluble NgR-Fc fusion protein is sNgR310-Fc, which comprises Fc joined to the C-terminal end of a soluble polypeptide having SEQ ID NO:5.

The soluble Nogo receptor polypeptide used in the practice of the methods of the invention may, in certain embodiments, comprise variants of SEQ ID NO:3, SEQ ID NO:4, SEQ ID NO:5, or SEQ ID NO:6, having up to 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, or more conservative amino acid substitutions. Methods for producing polypeptide variants having a number of conservative amino acid substitutions relative to the amino acid sequence of a reference polypeptide are known in the art. Exemplary/preferred amino acid substitutions are set forth in Table 3.

TABLE 3

| Original | | Preferred |
|---|---|---|
| Residue | Exemplary Substitutions | Substitutions |
| Ala (A) | val; leu; ile | val |
| Arg (R) | lys; gln; asn | lys |
| Asn (N) | gln; his; asp; lys; arg | gln |
| Asp (D) | glu; asn; | glu |
| Cys (C) | ser; ala | ser, ala |
| Gln (Q) | asn; glu | asn |
| Glu (E) | asp; gln | asp |
| Gly (G) | ala | ala |
| His (H) | asn, gln, lys, arg | arg |
| Ile (I) | leu; val; met; ala; phe; norleucine | leu |
| Leu (L) | norleucine; ile; val; met; ala; phe | ile |
| Lys (K) | arg; gln; asn | arg |
| Met (M) | leu; phe; ile | leu |
| Phe (F) | leu; val; ile; ala; tyr | tyr |
| Pro (P) | ala | ala |
| Ser (S) | thr | thr |
| Thr (T) | ser | ser |
| Tpr (W) | tyr; phe | tyr |
| Tyr (Y) | trp; phe; thr; ser | phe |
| Val (V) | ile; leu; met; phe; ala; norleucine | leu |

Specific soluble Nogo receptor polypeptides for use in the methods of the present invention include soluble NgR1 polypeptides with amino acid substitutions of individual cysteine residues. Any heterologous amino acid may be substituted for a cysteine in the polypeptides of the invention. Which different amino acid is used depends on a number of criteria, for example, the effect of the substitution on the conformation of the polypeptide fragment, the charge of the polypeptide fragment, or the hydrophilicity of the polypeptide fragment. In certain embodiments, the cysteine is substituted with a small uncharged amino acid which is least likely to alter the three dimensional conformation of the polypeptide, e.g., alanine, serine, threonine, preferably alanine. Cysteine residues that can substituted include C266, C309, C335 and C336. Making such substitutions through engineering of a polynucleotide encoding the polypeptide fragment is well within the routine expertise of one of ordinary skill in the art.

Exemplary soluble NgR-Fc fusion proteins with cysteine substitutions are Ala-Ala-human(h)NgR1-Fc which comprises Fc joined to the C-terminal end of a soluble polypeptide with the amino acid sequence of SEQ ID NO:23 and Ala-Ala-rat(r)NgR1-Fc which comprises Fc joined to the C-terminal end of a soluble polypeptide with the amino acid sequence of SEQ ID NO:24. (See Table 2).

### Antibodies

The methods of the invention may be performed using an antibody or an antigen-binding fragment thereof that specifically binds an immunogenic Nogo receptor-1 polypeptide and inhibits the binding of Nogo receptor-1 to a ligand (e.g., NogoA, NogoB, NogoC, MAG, OM-gp). Alternatively, the methods of the invention can be performed using an antibody specific for NogoA, NogoB, NogoC, MAG or OM-gp. The antibody or antigen-binding fragment used in the methods of the invention may be produced in vivo or in vitro. In some embodiments, the anti-Nogo receptor-1 antibody or antigen-binding fragment thereof is murine or human. In some embodiments, the anti-Nogo receptor-1 antibody or antigen-binding fragment thereof is recombinant, engineered, humanized and/or chimeric. In some embodiments, the antibody is selected from the antibodies described in International Patent Application No. PCT/US03/25004. Antibodies useful in the present invention may be employed with or without modification.

Exemplary antigen-binding fragments of the antibodies which may be used in the methods of the invention are Fab, Fab′, F(ab′)₂, Fv, Fd, dAb, and fragments containing complementarity determining region (CDR) fragments, single-chain antibodies (scFv), chimeric antibodies, diabodies and polypeptides that contain at least a portion of an immunoglobulin that is sufficient to confer specific antigen-binding to the polypeptide (e.g., inmmunoadhesins).

As used herein, Fd means a fragment that consists of the V_{H} and C_{HI} domains; Fv means a fragment that consists of the V_{L} and V_{H} domains of a single arm of an antibody; and dAb means a fragment that consists of a V_{H} domain (Ward et al., Nature 341:544-46 (1989)). As used herein, single-chain antibody (scFv) means an antibody in which a V_{L} region and a V_{H} region are paired to form a monovalent molecules via a synthetic linker that enables them to be made as a single protein chain (Bird et al., Science 242:423-26 (1988) and Huston et al., Proc. Natl. Acad. Sci. USA 85:5879-83 (1988)). As used herein, diabody means a bispecific antibody in which V_{H} and V_{L} domains are expressed on a single polypeptide chain, but using a linker that is too short to allow for pairing between the two domains on the same chain, thereby forcing the domains to pair with complementary domains of another chain and creating two antigen-binding sites (see, e.g., Holliger et al., Proc. Natl. Acad. Sci. USA 90:6444-48 (1993) and Poljak et al., Structure 2:1121-23 (1994)).

### Immunization

Antibodies for use in the methods of the invention can be generated by immunization of a suitable host (e.g., vertebrates, including humans, mice, rats, sheep, goats, pigs, cattle, horses, reptiles, fishes, amphibians, and in eggs of birds, reptiles and fish). Such antibodies may be polyclonal or monoclonal. For a review of methods for making antibodies see, e.g., Harlow and Lane (1988), Antibodies, A Laboratory Manual; Yelton et al., Ann. Rev. of Biochem., 50:657-80 (1981); and Ausubel et al. (1989), Current Protocols in Molecular Biology (New York: John Wiley & Sons). Determination of immunoreactivity with an immunogenic Nogo receptor polypeptide may be made by any of several methods well known in the art, including, e.g., immunoblot assay and ELISA. Monoclonal antibodies for use in the methods of the invention can be made by standard procedures as described, e.g., in Harlow and Lane (1988), supra.

For example, a host may be immunized with an immunogenic Nogo receptor-1 polypeptide either with or without an adjuvant. Suitable polypeptides are described in, for example, International Patent Applications PCT/US01/31488, PCT/US02/32007 and PCT/US03/25004. The host also may be immunized with Nogo receptor-1 associated with the cell membrane of an intact or disrupted cell and antibodies identified by binding to a Nogo receptor-1 polypeptide. Other suitable techniques for producing an antibody involve in vitro exposure of lymphocytes to the Nogo receptor-1 or to an immunogenic polypeptide of the invention, or alternatively, selection of libraries of antibodies in phage or similar vectors. See Huse et a., Science 246:1275-81 (1989).

Anti-Nogo receptor-1 antibodies used in the methods of this invention also can be isolated by screening a recombinant combinatorial antibody library. Methodologies for preparing and screening such libraries are known in the art. There are commercially available methods and materials for generating phage display libraries (e.g., the Pharmacia Recombinant Phage Antibody System, catalog no. 27-9400-01; the Stratagene SurfZAP™ phage display kit, catalog no. 240612; and others from MorphoSys). Following screening and isolation of an anti-Nogo receptor-1 antibody from a recombinant immunoglobulin display library, the nucleic acid encoding the selected antibody can be recovered from the display package (e.g., from the phage genome) and subcloned into other expression vectors by standard recombinant DNA techniques. To express an antibody isolated by screening a combinatorial library, DNA encoding the antibody heavy chain and light chain or the variable regions thereof is cloned into a recombinant expression vector and introduced into a host cell.

Monoclonal anti-NgR1 antibodies were generated as described previously. Li, W. et al., J. Biol. Chem. 279:43780-43788-(2004). The antigens used were srNgR310-Fc (Li, S. et al., J. Neurosci. 24: 10511-10520 (2004)) and COS-7 cells expressing rat NgR1. The monoclonal antibody, 1D9, was characterized by ELISA binding assays and FACS analysis. 1D9 binds only to rat NGR1 and does not recognize human or mouse NgR1, nor NgR2 and NgR3. Fab fragments were purified according to standard procedures and found to have minimal effect on rhoA activation on primary neurons. Monoclonal antibodies which bind to human NgR1 are described, e.g., in PCT Publication No. WO 2005/016955 A2, which is incorporated herein by reference in its entirety.

### Uses for Nogo Receptor Antagonists

This invention relates to methods of treating conditions involving neuronal degeneration, including degeneration of sensory neurons such as retinal ganglion cells and hairy cells. For example, the present invention relates to a method of promoting regeneration or survival of a sensory neuron in a mammal displaying signs or symptoms of a condition involving neuronal cell death, comprising administering to the mammal a therapeutically effective amount of a Nogo receptor-1 antagonist of the present invention. In certain embodiments, the present invention relates to a method of promoting regeneration or survival of a sensory neuron in a mammal displaying signs or symptoms of a condition involving neuronal cell death, without promoting CNS neurite outgrowth, comprising administering to the mammal a therapeutically effective amount of a Nogo receptor-1 antagonist of the present invention which promotes neuronal survival or regeneration but does not promote neurite outgrowth.

The invention includes methods for treating optical neuropathies including, but not limited to, e.g., glaucoma, optic nerve sheath meningioma and glioma, Graves' ophthalmopathy, benign or malignant orbital tumors, metastatic lesions, tumors arising from the adjacent paranasal sinuses or middle cranial fossa, giant pituitary adenomas, brain tumors or abscesses, cerebral trauma or hemorrhage, meningitis, arachnoidal adhesions, pseudotumor cerebri, cavernous sinus thrombosis, dural sinus thrombosis, encephalitis, space-occupying brain lesions, severe hypertensive disease or pulmonary emphysema, ischemic optic neuropathy (including anterior ischemic optic neuropathy), retinal blood vessel occlusion, diabetic retinophathy, macular degeneration, retinitis pigmentosa and Leber's disease (See, e.g., U.S. Pat. No. 6,162,428 and U.S. Patent Appl. Nos. 20040228795 and 20040192699).

In some embodiments of this invention, the RGC degeneration or death is associated with a disease, disorder or condition including, but not limited to, glaucoma. In some embodiments of this invention, the hairy cell degeneration or death is associated with a disease, disorder or condition including, but not limited to, hearing loss, including age-related hearing loss.

### Nogo Receptor Antagonist Pharmaceutical Compositions

The Nogo receptor antagonists used in the methods of the invention may be formulated into pharmaceutical compositions for administration to mammals, including humans. The pharmaceutical compositions used in the methods of this invention comprise pharmaceutically acceptable carriers.

Pharmaceutically acceptable carriers useful in these pharmaceutical compositions include, e.g., ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The compositions used in the methods of the present invention may be administered by any suitable method, e.g., parenterally, intraventricularly, orally, by inhalation spray, topically, rectally, nasally, buccally, vaginally, via an implanted reservoir, e.g., a capsule implant (See, for e.g., Sieving et al., PNAS 103(10):3896-3901 (2006)) or contact lens (See, for e.g., U.S. Pat. No. 6,410,045 and Gulsen, D. and Chauhan A., Invest Ophthalmol Vis Sci. 45(7):2342-2347 (2004)). The term “parenteral” as used herein includes subcutaneous, intravenous, intramuscular, intra-articular, intra-synovial, intrasternal, intrathecal, intrahepatic, intralesional and intracranial injection or infusion techniques. In methods of the invention, the Nogo receptor antagonist must have access to the eye. Where the Nogo receptor antagonist is a soluble Nogo receptor or anti-Nogo receptor antibody the antagonist is generally administered in eyedrops or intraocularly, e.g., intravitreally. Where the Nogo receptor antagonist is a molecule that may gain access to the eye after delivery to other distant sites, the route of administration may be by one or more of the various routes described below.

Sterile injectable forms of the compositions used in the methods of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Parenteral formulations may be a single bolus dose, an infusion or a loading bolus dose followed with a maintenance dose. These compositions may be administered once a day or on an “as needed” basis.

Certain pharmaceutical compositions used in the methods of this invention may be orally administered in any orally acceptable dosage form including, e.g. capsules, tablets, aqueous suspensions or solutions. Certain pharmaceutical compositions also may be administered by nasal aerosol or inhalation. Such compositions may be prepared as solutions in saline, employing benzyl alcohol or other suitable preservatives, absorption promoters to enhance bioavailability, fluorocarbons, and/or other conventional solubilizing or dispersing agents.

The amount of Nogo receptor antagonists that may be combined with the carrier materials to produce a single dosage form will vary depending upon the host treated and the particular mode of administration. The composition may be administered as a single dose, multiple doses or over an established period of time in an infusion. Dosage regimens also may be adjusted to provide the optimum desired response (e.g., a therapeutic or prophylactic response).

The methods of the invention use a “therapeutically effective amount” or a “prophylactically effective amount” of a Nogo receptor antagonist. A therapeutically or prophylactically effective amount of the Nogo receptor antagonist used in the methods of the invention may vary according to factors such as the disease state, age, sex, and weight of the individual. A therapeutically or prophylactically effective amount is also one in which any toxic or detrimental effects are outweighed by the therapeutically beneficial effects.

A specific dosage and treatment regimen for any particular patient will depend upon a variety of factors, including the particular Nogo receptor antagonist, the patient's age, body weight, general health, sex, and diet, and the time of administration, rate of excretion, drug combination, and the severity of the particular disease being treated. Judgment of such factors by medical caregivers is within ordinary skill in the art. The amount of antagonist will also depend on the individual patient to be treated, the route of administration, the type of formulation, the characteristics of the compound used, the severity of the disease, and the desired effect. The amounts of antagonists can be determined by pharmacological and pharmacokinetic principles well-known in the art.

In the methods of the invention, the Nogo receptor antagonists are generally administered intraocularly, e.g. intravitreally. Compositions for administration according to the methods of the invention can be formulated so that a dosage of 0.001-10 mg/kg body weight per day of the Nogo receptor antagonist is administered. In some embodiments of the invention, the dosage is 0.01-1.0 mg/kg body weight per day. In some embodiments, the dosage is 0.05-0.5 mg/kg body weight per day.

Supplementary active compounds also can be incorporated into the compositions used in the methods of the invention. For example, a Nogo receptor antibody or an antigen-binding fragment thereof, or a soluble Nogo receptor polypeptide or a fusion protein may be coformulated with and/or coadministered with one or more additional therapeutic agents.

The compositions may also comprise a Nogo receptor antagonist dispersed in a biocompatible carrier material that functions as a suitable delivery or support system for the compounds. Suitable examples of sustained release carriers include semipermeable polymer matrices in the form of shaped articles such as suppositories or capsules. Implantable or microcapsular sustained release matrices include polylactides (U.S. Pat. No. 3,773,319; EP 58,481), copolymers of L-glutamic acid and gamma-ethyl-L-glutamate (Sidman et al., Biopolymers 22:547-56 (1985)); poly(2-hydroxyethyl-methacrylate), ethylene vinyl acetate (Langer et al., J. Biomed. Mater. Res. 15:167-277 (1981); Langer, Chem. Tech. 12:98-105 (1982)), poly-D-(-)-3hydroxybutyric acid (EP 133,988) or a membrane composed of polyether sulfone, caprolactame and polyvinyl pyrrolidone (Tao et al., Invest. Ophthamol. Vis. Sci. 43:3292-3298 (2002)).

### EXAMPLES

### Example 1

### NgR1 Is Expressed In Rat Retinal Ganglion Cells

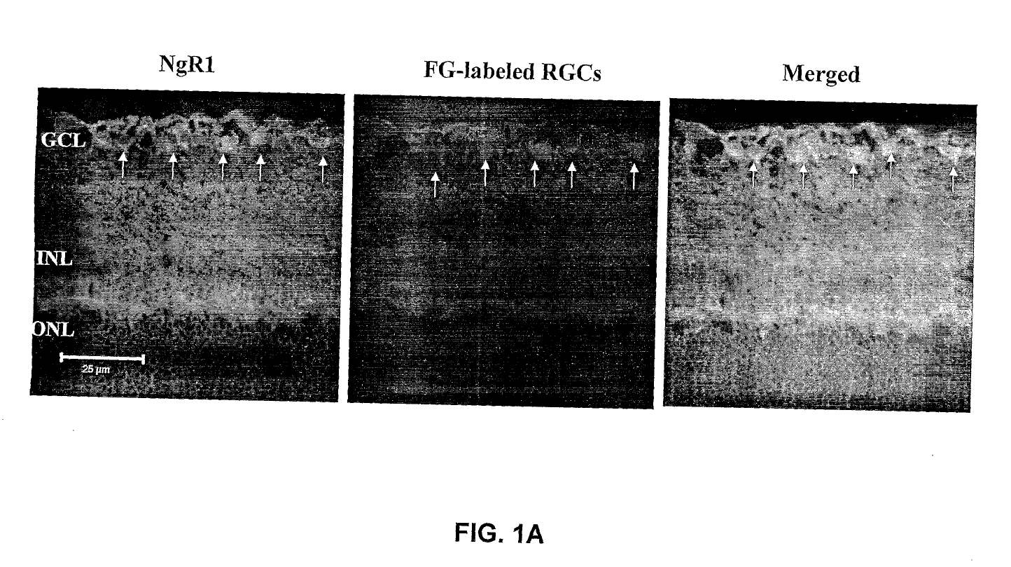

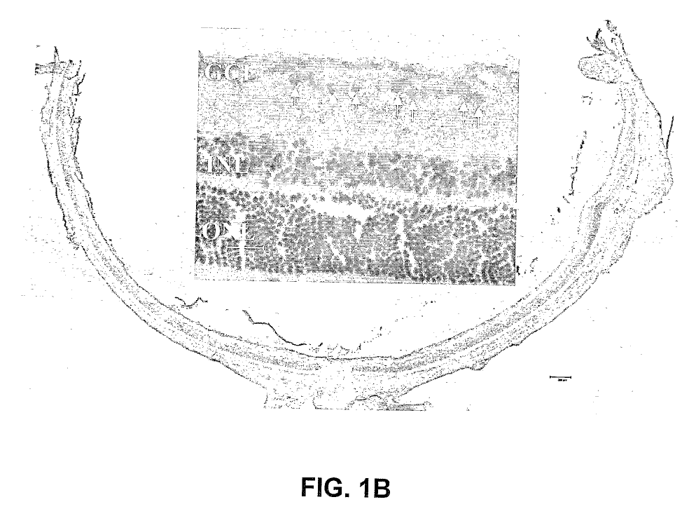

Immunohistochemistry shows that NgR1 is expressed in rat retinal ganglion cells (RGCs). The optic nerve of young female Spargue Dawley rat was transected intraorbitally 1.5 mm away from the optic disc. A piece of gelfoam soaked with 6% fluoro-gold was applied to the newly transected optic nerve right behind the optic disc to label the surviving RGCs. After 2 days, the animal was sacrificed, the retina fixed in 4% paraformaldehyde and embedded in paraffin. Immunohistochemistry was performed on the retinal sections using the murine monoclonal anti-NgR1 antibody, 1D9; and detected by a Texas red conjugated secondary antibody directed against mouse IgG. The sections were examined under a fluorescence microscope. NGR1 staining appears in red and Fluoro-Gold staining appears in blue. FIGS. 1A-1B.

### Example 2

### Binding of a Monoclonal Anti-NgR1 Antibody, 1D9, to Soluble Rat Nogo Receptor 310 (srNgR310)

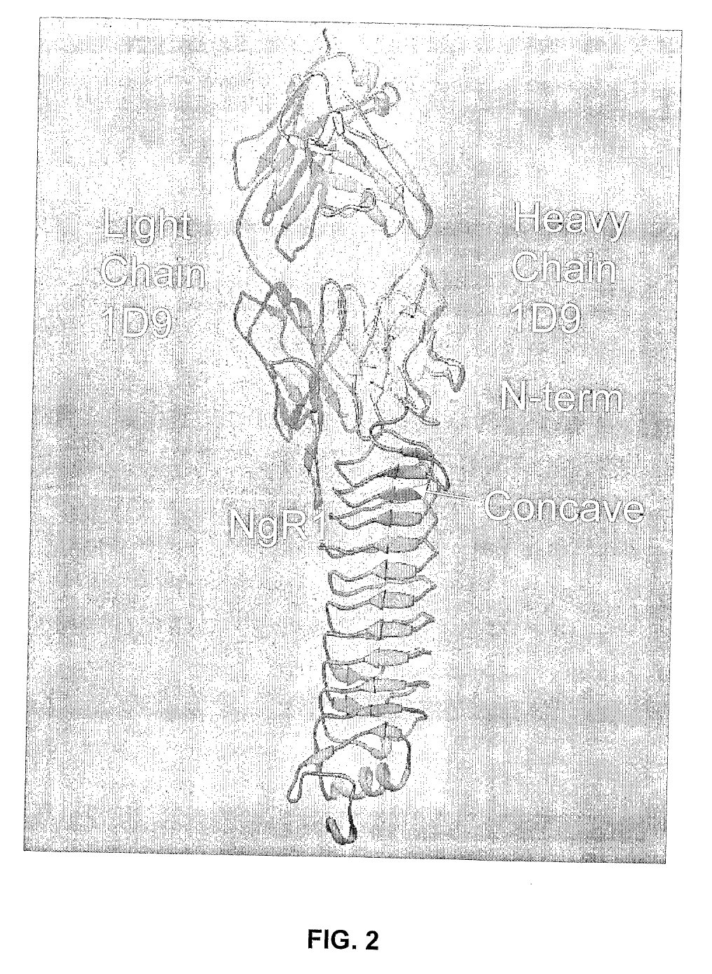

Structural analyses performed on the co-crystal complex of the 1D9 Fab and a soluble fragment of rat NgR1 (srNgR310) shows that this antibody binds near the junction of the N-terminus cap and leucine rich repeat domain on rat NgR1. FIG. 2. For crystallization of rat srNgR310-Fc with the 1D9 Fab, each macromolecule was cleaved with papain and purified from the Fc portion and stored in 10 mM Hepes pH 7, 50 mM NaCl. The complex was prepared at 80 μM each and mixed at a volumetric ratio of 2:1 with a reservoir solution consisting of 14% Peg3350, 0.4 M Zinc Acetate, 0.1M Magnesium Chloride. The solution was incubated at 20 C for 1 hr and centrifuged at 12,000×g for 3 minutes to remove precipitate. Crystals were grown by placing 3-5 uL of the supernatant over wells containing 50% to 100% of the reservoir solution at 20 C. Thin plate-like crystals grew over a period of 1 week at 20 C. The crystals were cryoprotected by quickly transferring into 0.2 M Zinc Acetate, 8% Peg3350, 25% Ethylene Glycol for 2 min and then frozen by quick transfer into liquid nitrogen.

Crystals approximately 10 μm thick diffracted to 3.2 Å at beamline ×25 at the National Synchrotron Light Source (Upton, N.Y.). Data processing with the HKL program package v. 1.97 (Otwinowski, Z., and Minor, W. Processing X-ray diffraction data collected in oscillation mode. Methods Enzymol 276: 307-326 (1997)) revealed the crystals to belong to a P21212 space group and approximate cell dimensions a=90.6 Å, b=188.6 Å, c=125.5 Å, and α=β=γ=90, consistent with 2 Fab-NgR1 complexes per asymmetric unit.

The crystal structure was solved by utilizing information on multiple isomorphous replacement experiments on soaked crystals to identify common mercury sites bound to the NgR along with molecular replacement. The space group was identified by inspection of mercury and gold isomorphous difference patterson maps in which a consistent 5 sigma peak was identified at the w=0 harker section. Molecular replacement with MOLREP (Vagin, A., and Teplyakov, A. MOLREP: an automated program for Molecular replacement. J. Appl. Cryst. 30: 1022-1025 (1997)) utilizing a rat NgR homology model based on the human NgR1 structure (pdb code 1OZN) (He, X. L. et al., Neuron 38: 177 (2003)) and a homology model for the 1D9 Fab led to placement of one NgR1, one Fab and a second NGR1 molecules with a resulting R-factor of 48% and clear density for the CDR regions of the Fab. The placement of the model was confirmed by mapping the mercury sites identified from difference patterson maps onto equivalent positions on both NgR1 molecules near Asp138 and His182. No additional Fab fragments have been clearly identified in the density. Refinement of the two NgR1 and 1 Fab using CNX (Brunger, A. T. et al., Acta Crystallogr D Biol Crystallogr 54: 905-921 (1998)) to 3.2 Å resolution has proceeded with a current R-factor of 42% and Rfree of 46%.

Table 4 shows the contacts between the 1D9 Fab and rat NGR1. Contacts in which atoms from the Fab are within 3.9 A distance from atoms in rat NgR1 are listed and those contacts that could form a hydrogen bond with either the main chain or side chain have an associated asterisk(*).

TABLE 4

| CDR L1 | CDR L2 | CDR L3 |
|---|---|---|
| KSSQSLLNSRNRKNYLA | WASTRES | MQSYNLFT |
| N31-1D9 | | |
| Y71-NgRI | | |
| R33-1D9 | | |
| Y71, D97*, A94-NgRI | | |
| N34-1D9 | | |
| S70*, Y71-NgRI | | |

| CDR H1 | CDR H2 | CDR H3 |
|---|---|---|
| GFSLSSYGVH | VIWSGGNTHYNSALMS | VGIYYEGAWFAY |
| F27-1D9 | S53-1D9 | G99-1D9 |
| P26-NgRI | S79*-NgRI | A53-NgRI |
| S28-1D9 | G54-1D9 | Y101-1D9 |
| P26-NgRI | R81-NgRI | P73*, A74*, S76*, |
| | | A50, V51*-NgRI |
| S30-1D9 | N56-1D9 | Y102-1D9 |
| A57*-NgRI | Q78*-NgRI | Y71, P73, A50, |
| | | V51*, L36*-NgRI |
| S31-1D9 | | E103-1D9 |
| G54-NgRI | | Q49, A50, V51, P52, |
| | | A53*-NgRI |
| Y32-1D9 | | G104-1D9 |
| P26, P28-NgRI | | A53-NgRI |

*indicates H-bond interactions

### Example 3

### Soluble Nogo Receptor (310)-Fc Promotes Neuronal Survival In Vitro

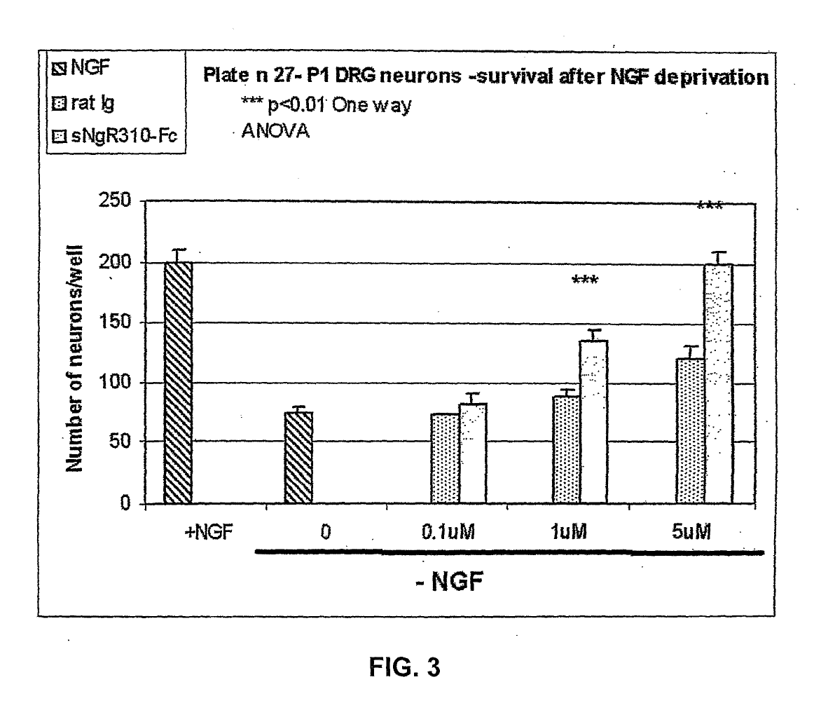

P1 or P2 rat dorsal root ganglia (DRG) neurons were isolated from adult female Sprague-Dawley rats and plated in 96-well culture plates with Ham's F12 culture medium containing 5% heat-inactivated donor horse serum (JRH Bioscience, Logan, Utah), 5% heat-inactivated FBS (JRH Bioscience) and 50 ng/ml murine nerve growth factor (JRH Bioscience). After plating, nerve growth factor (NGF) was removed from the culture media and the cells were kept for 7 days in the presence or absence of increasing concentrations of srNgR310-Fc (or rat IgG as control). The cells were fixed and stained for viable nuclei. The results this experiment indicated that srNgR310-Fc promoted survival of DRG neurons under NGF deprivation (FIG. 3).

In separate experiments, cortical neurons were isolated from rat embryos (embryonic day 18) and plated in 96-well culture plates. After plating, nerve growth factor (NGF) was removed from the culture media and the cells were kept for 7 days in the presence or absence of srNgR310-Fc. The cells were fixed and stained for viable nuclei. The results of this experiment indicated that srNgR310-Fc also promoted survival of cortical neurons under NGF deprivation (Table 5).

TABLE 5
Number of Viable DRG Neurons

| | | Without NGF/With 5 μM |
|---|---|---|
| With NGF | Without NGF | srNgR310-Fc |
| 25.2 ± 2.4 | 2.5 ± 0.3 | 11.1 ± 2.7 |

### Example 4

### srNgR310-Fc Promotes Survival of Retinal Ganglion Cells in Vivo

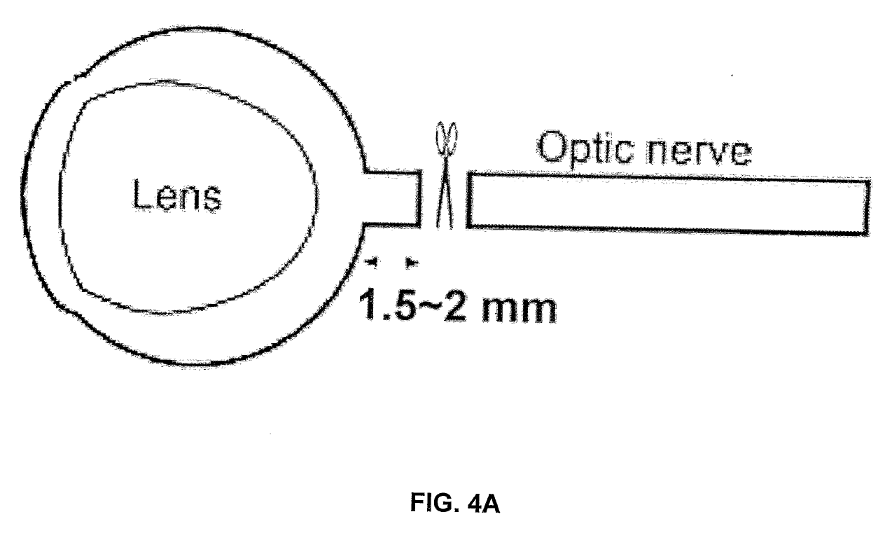

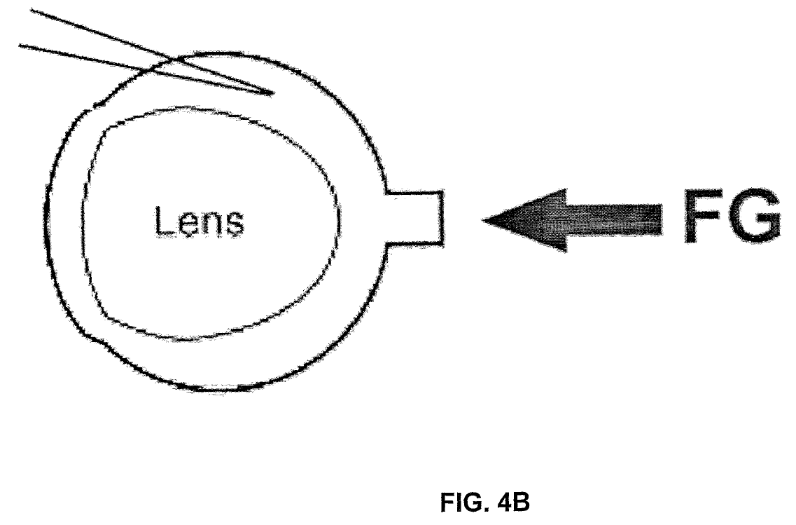

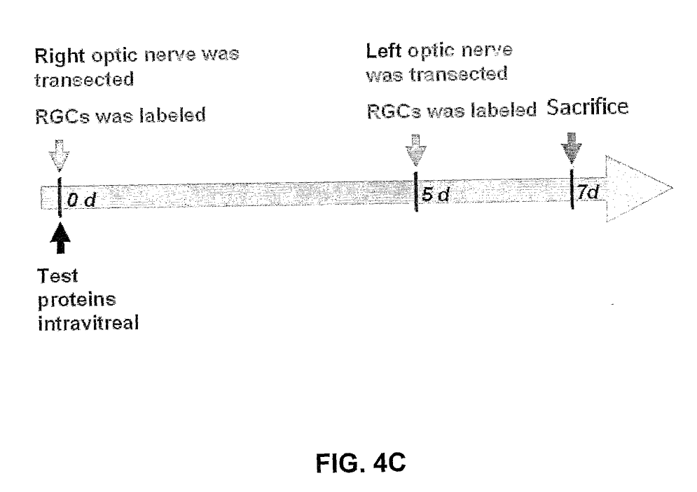

We confirmed in vivo that srNgR310-Fc promotes neuronal survival using the optic nerve transection model (FIG. 4A-4C). The right optic nerve of young adult female Sprague Dawley rats was transected intraorbitally 1.5 mm from the optic disc (FIG. 4A). A piece of gelfoam soaked with 6% Fluoro-Gold (FG) was applied to the newly transected site right behind the optic disc to label the surviving RGCs. (FIG. 4B). The animals were divided into 2 groups (n=6 in each group) receiving either 2 μg srNgR310-Fc or PBS by intravitreal injection (4 μl ) (FIG. 4C). The intravitreal injections were performed immediately after the optic nerve transection.

On day 5, the left optic nerve of each animal was transected and 6% FG were used to label the surviving RGCs to serve as the internal control. Animals were sacrificed with an overdose of Nembutal and the retinas dissected in 4% paraformaldehyde. Four radial cuts were made to divide the retinas into four quadrants (superior, inferior, nasal and temporal). The retinas were then post-fixed in the same fixative for 1 hour before they were flat-mounted with the mounting medium (Dako). FIG. 4B. The slides were examined under a fluorescence microscope using an ultra-violet filter (excitation wavelength=330-380 nm). Labeled RGCs were counted along the median line of each quadrants starting from the optic disc to the peripheral border of the retina at 500 mm intervals, under an eyepiece grid of 200×200 mm. The percentage of surviving RGCs resulting from each treatment was expressed by comparing the number of surviving RGCs in the injured eye with the contralateral eye. All data were expressed as mean±SEM. Statistical significance was evaluated by one way ANOVA, followed by a Tukey-Kramer post hoc test. Differences were considered significant for p<0.05.

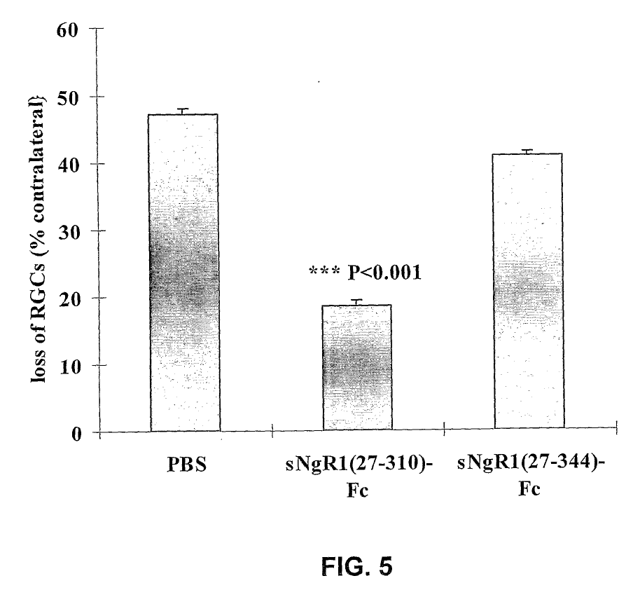

The data indicated that srNR310-Fc treated animals showed significant neuronal survival (approximately 80%) when compared to PBS-treated animals, which each only showed approximately 50% neuronal survival (FIG. 5). These results confirmed that the NgR antagonist also promoted neuronal survival in vivo.

### Example 5

### srNgR310-Fc Promotes Survival of Retinal Ganglion Cells in a Rat Glaucoma Model 1

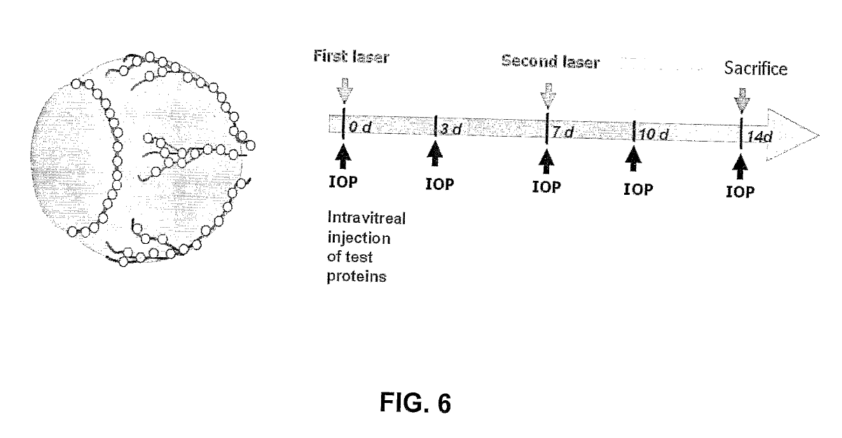

We tested srNgR310-Fc in an established glaucoma model (Woldemussie et al., “Neuroprotection of retinal ganglion cells by brimonidine in rats with laser-induced chronic ocular hypertension,” Invest Ophthalmol. Vis. Sci. 42, 2849-2855 (2001)). In this model, an argon laser is used to block the outflow of aqueous humor by photocoagulation of the limbal and episcleral drainage vessels, resulting in reliable increase of intraocular pressure (IOP) (See FIG. 6).

The experiments were performed in adult female Sprague-Dawley rats weighing approximately 250-280 g. Just prior to the first laser operation, baseline IOP was measured using a tonometer to obtain the normal IOP reading. Elevated IOP was induced in the right eye by laser photocoagulation of the episcleral and limbal veins at a 270° arc around the peri-limbal region. The laser treatments were conducted twice at 7 days intervals. Around 20 and 60 laser irradiation (1W, 0.1 sec, spot size: 50-100 mm) were applied onto the episcleral veins and the limbal veins, respectively. The IOP was monitored 3 days after each operation to confirm that IOP was elevated in the laser-treated eye. 2 μg/eye srNgR310-Fc or PBS was injected intra-orbitally immediately after the first laser coagulation. When the second laser operation was performed, a piece of gelfoam soaked with 6% Fluoro-Gold was applied to the superior colliculi to retrogradely label the RGCs. Three days after the second laser operation, the animals were sacrificed and the retinas were flat-mounted.

The number of RGCs was counted in three quadrants (nasal, inferior & temporal) of all the retinas. The changes in the densities of RGCs were expressed as percentage loss of RGCs by comparing the laser-treated eyes with the contralateral, control eye of the same animal. Consistent elevation of IOP was observed in all the laser-operated eyes.

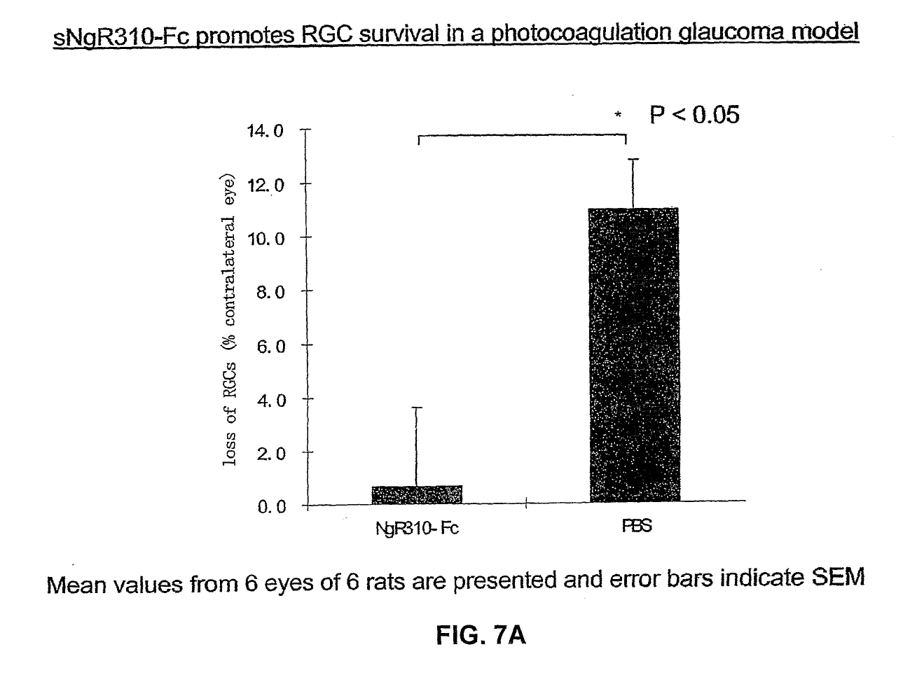

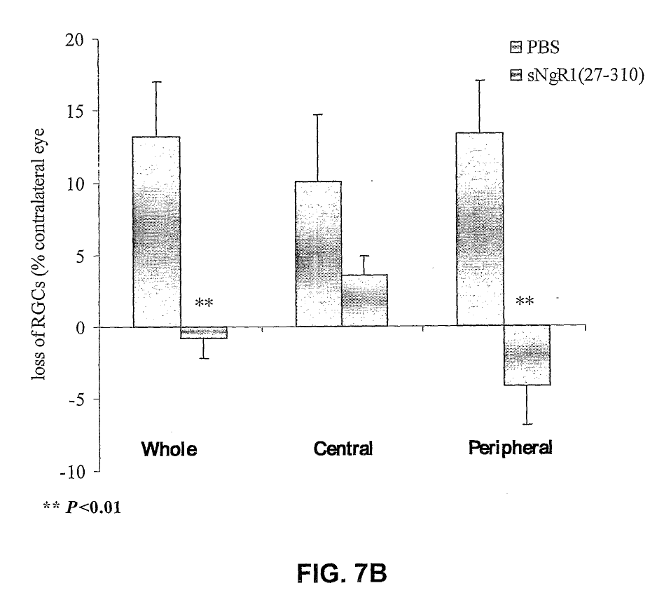

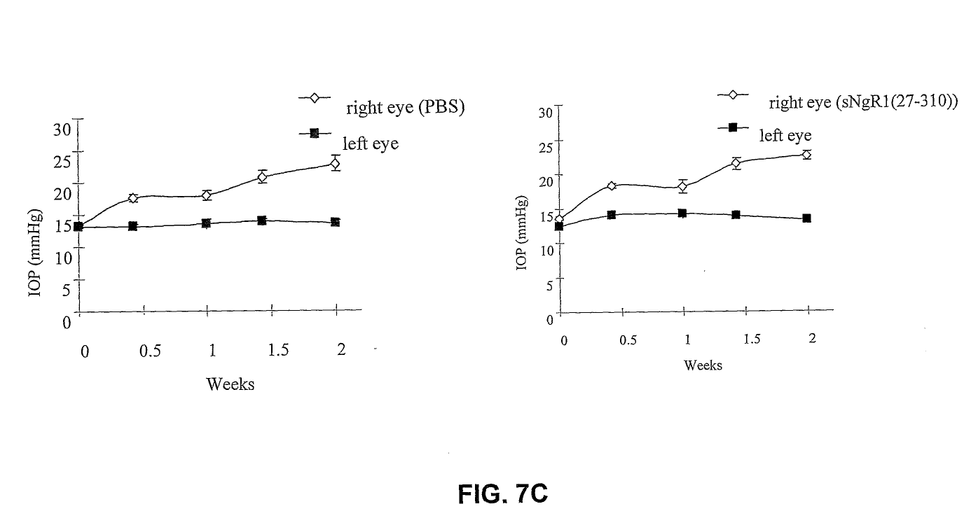

The IOP was 1.5 times higher in the laser-operated eye compared to the sham control left eye. The data indicated that a single intravitreal injection of srNgR310-Fc promoted RGC survival in the eyes with elevated IOP (FIG. 7A).

Another experiment confirmed these results. In this experiment, treatment with srNgR310-Fc enhanced the survival of RGCs in the entire and peripheral retina compared to the PBS control after laser treatment while treatment with sNgR1(27-310)-Fc had no effect in lowering the IOP after laser treatment (P-value represent comparison to the PBS group) (FIGS. 7B-7C).

### Example 6

### Anti-NgR1 Antibody, 1D9, Promotes Neuronal Survival In Vitro

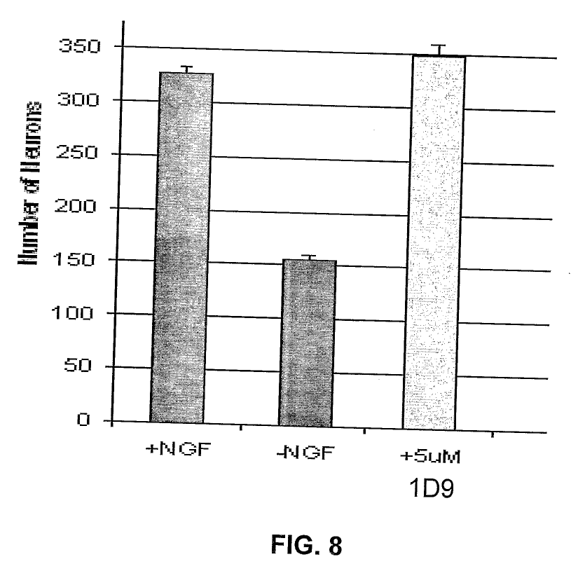

P1 or P2 rat dorsal root ganglia (DRG) neurons were isolated from adult female Sprague-Dawley rats and plated in 96-well culture plates with Ham's F12 culture medium containing 5% heat-inactivated donor horse serum (JRH Bioscience, Logan, Utah), 5% heat-inactivated FBS (JRH Bioscience) and 50 ng/ml murine nerve growth factor (JRH Bioscience). After plating, nerve growth factor (NGF) was removed from the culture media and the cells were kept for 6 days in the presence of 1D9. Surviving cells were fixed, stained for β-III tubulin, and counted using Cellomics Arrayscan. The results of this experiment indicated that 1D9 treatment protected serum deprived rat p2 DRG neurons for 6 days in culture (FIG. 8). At least two experiments with similar results were obtained. Similar results were obtained for cerebellar neurons (data not shown).

### Example 7

### A Monoclonal Anti-NgR1 Antibody, Promotes Survival of Retinal Ganglion Cells In Vivo

The ability of a chimeric monoclonal anti-NgR1 antibody Fab fragment (1D9 Fab), to promote neuronal survival was assayed using the optic nerve transection model. The experimental protocol used in this example is similar to that which is described in Example 4. Briefly, chimeric Fab was given intravitrealy (2 μg per eye) to optic nerve transected rats. Retinal ganglion cell survival was monitored. The results showed that 1D9 treatment results in enhanced survival of retinal ganglion cells after acute or chronic injuries.

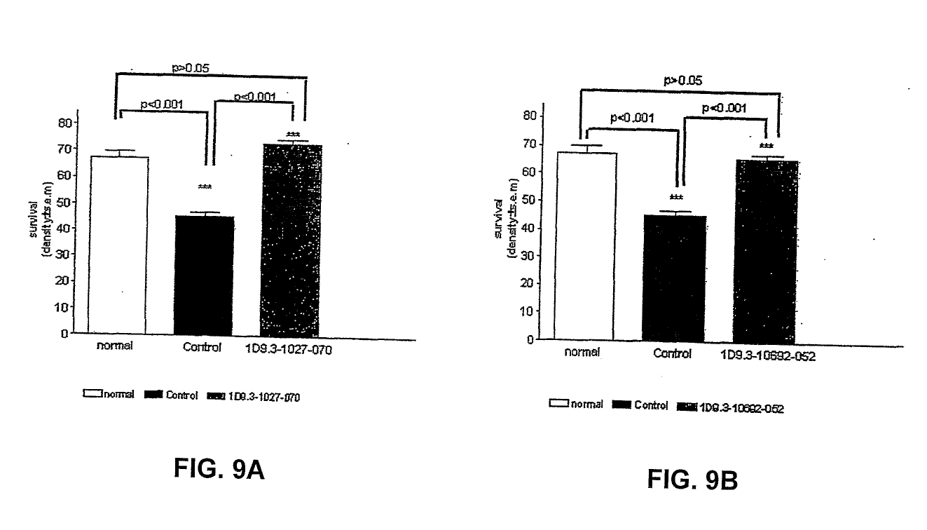

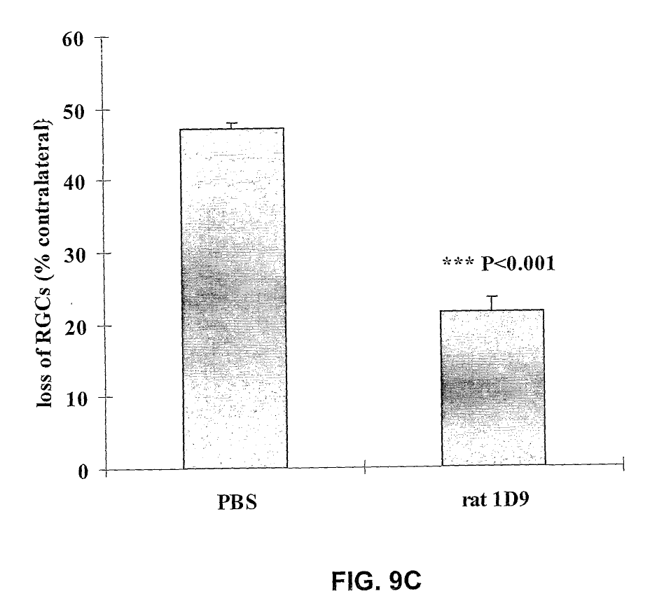

The direct administration of two separate preparations of 1D9 Fab (designated 1D9.3-1027.070 and 1D9.3-10692.052, respectively) by intravitreal injection in rats caused significant improvement in RGC survival compared to control experiments in which buffer alone was administered (FIGS. 9A-C).

### Example 8

### A Monoclonal Anti-NgR1 Antibody, 1D9, Promotes Survival of Retinal Ganglion Cells in a Rat Glaucoma Model

We tested the anti-NgR1 antibody Fab fragment (1D9 Fab) in an established glaucoma model (Woldemussie et al., “Neuroprotection of retinal ganglion cells by brimonidine in rats with laser-induced chronic ocular hypertension,” Invest Ophthalmol. Vis. Sci. 42, 2849-2855 (2001)). As described above, an argon laser is used to block the outflow of aqueous humor by photocoagulation of the limbal and episcleral drainage vessels, resulting in reliable increase of intraocular pressure (IOP).

The experiments were performed in adult female Sprague-Dawley rats weighing approximately 250-280 g. Just prior to the first laser operation, baseline IOP was measured using a tonometer to obtain the normal IOP reading. Elevated IOP was induced in the right eye by laser photocoagulation of the episcleral and limbal veins at a 270° arc around the peri-limbal region. The laser treatments were conducted twice at 7 days intervals. Around 20 and 60 laser irradiation (1 W, 0.1 sec, spot size: 50-100 mm) were applied onto the episcleral veins and the limbal veins, respectively. The IOP was monitored 3 days after each operation to confirm that IOP was elevated in the laser-treated eye. 2 μg/eye 1D9 or PBS was injected intra-orbitally immediately after the first laser coagulation. Surviving RGC's were retrogradely labeled by placing a piece of Gelfoam soaked with 6% Fluorogold at the ocular stump at 2 days before sacrifice. At predetermined time (7 and 14 days postoperation), the animals were killed and their eyes were dissected out and were flat mounted.

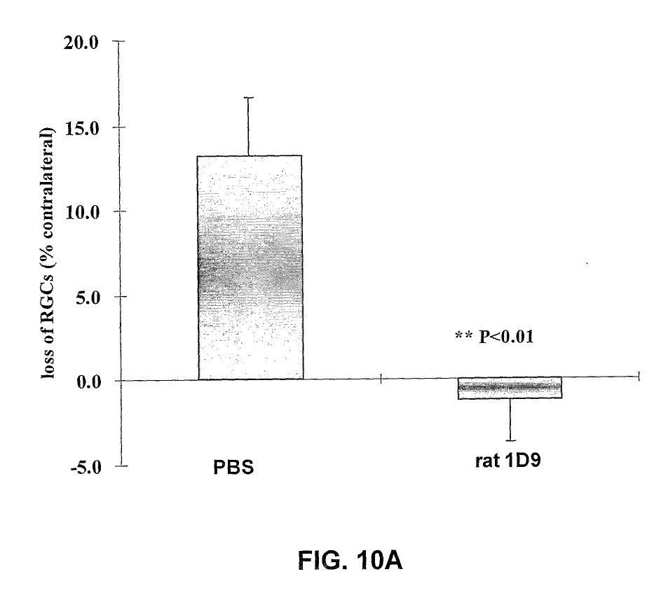

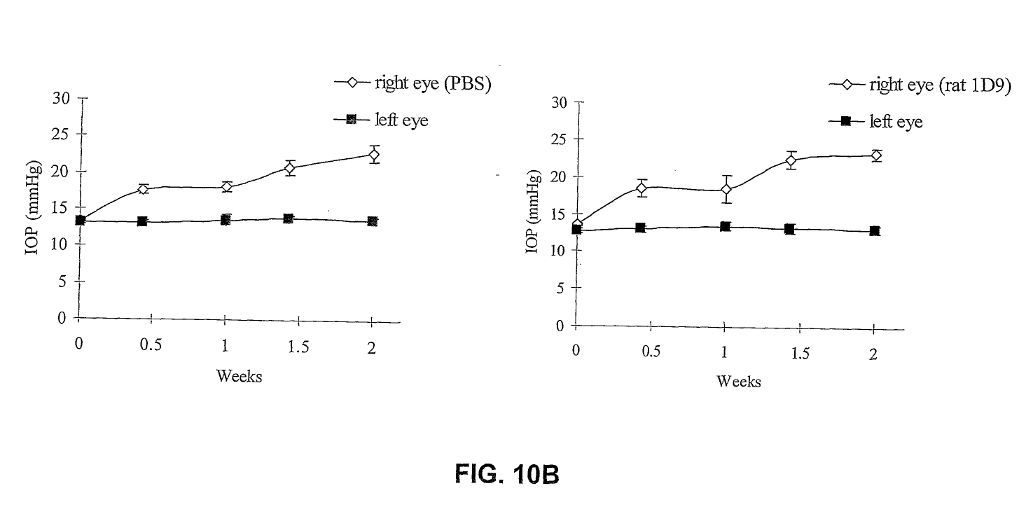

The number of surviving RGCs was counted under an eyepiece grid (200 μm×200 μm) at 500 μm intervals along the median of each quadrant from the optic disc to the peripheral border of the retina. Statistical significance was evaluated by one-way ANOVA, followed by Tukey-Kramer post hoc test. Differences were considered significant when P<0.05. The changes in the densities of RGCs were expressed as percentage loss of RGCs by comparing the laser-treated eyes with the contralateral, control eye of the same animal. Consistent elevation of IOP was observed in all the laser-operated eyes. An increase of about 1.7 times in IOP was observed in the laser-treated eyes in all groups. The data indicated that injection of 1D9 promoted RGC survival in the eyes after induction of ocular hypertension compared to the PBS group while 1D9 had no effect in lowering the IOP after laser treatment (FIGS. 10A-B).

### Example 9

### Ala-Ala-rNgR310-Fc and Ala-Ala-bNgR310-Fc Promote Survival of Retinal Ganglion Cells in a Rat Glaucoma Model

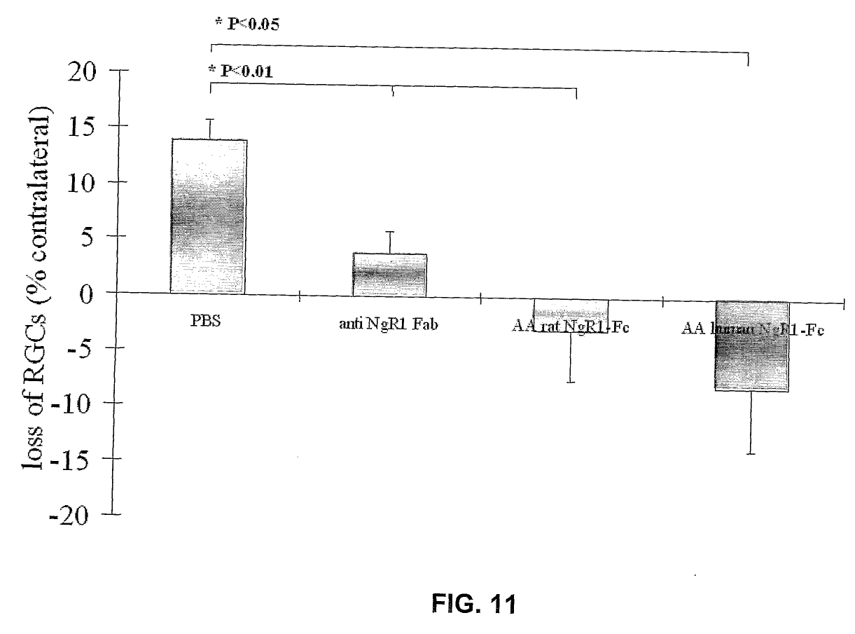

As described above, intraocular pressure in the right eye of a rat was artificially raised by Argon laser photocoagulation of the limbal and three episcleral veins. 2 μg/eye of Ala-Ala-rNgR310-Fc, Ala-Ala-hNgR310-Fc, the anti-NgR1 1D9 Fab or PBS was administered to the injured eye immediately after the injury by a single intravitreal injection [2 μg/2 μl per eye]. On day 7, a second laser photocoagulation procedure was performed to maintain the intraocular pressure in the eye. On day 10, a piece of gel foam soaked with 6% fluoro-gold was placed on the surface of the superior colliculus to retrograde label the retinal ganglion neurons. The animals were sacrificed on day 14, and the retinas were flat mounted and fixed. The number of surviving retinal ganglion neurons [defined as cell bodies showing fluorescence from the fluoro-gold] was counted under an eyepiece grid (200 μm×200 μm) at 500 μm intervals along the median line of each quadrant from the optic disc to the peripheral border of the retina. The total number of surviving retinal ganglion neurons of the right eye was normalized to that of the left eye and expressed as % loss of retinal ganglion neurons. Each experiment group contains 3-5 animals and statistics were performed using One-way ANOVA or t-test. The data indicated that injection of Ala-Ala-rNgR310-Fc, Ala-Ala-hNgR310-Fc or anti-NgR1 1D9 Fab promoted RGC survival in the eyes after induction of ocular hypertension compared to the PBS group (FIG. 11).

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A method of promoting regeneration or survival of a sensory neuron in a mammal displaying signs or symptoms of a condition involving neuronal cell death, comprising administering to the mammal a therapeutically effective amount of an NgR1 antagonist.

2. The method of claim 1 , wherein the sensory neuron is a hairy cell.

3. The method of claim 2, wherein the mammal suffers from hearing loss.

4. The method of claim 1, wherein the sensory neuron is a retinal ganglion cell (RGC).

5. The method of claim 4, wherein the NgR1 antagonist is administered directly into the eye.

6. The method of claim 5, wherein the NgR1 antagonist is administered intravitreally.

7. The method of claim 4, wherein the NgR1 antagonist is administered via a capsule implant.

8. The method of claim 4, wherein the mammal suffers from an optical neuropathy.

9. The method of claim 8, wherein said optical neuropathy is glaucoma.

10. The method of claim 1, wherein the NgR1 antagonist comprises a soluble form of a mammalian NgR1.

11. The method of claim 10, wherein the soluble form of a mammalian NgR1 comprises amino acids 26 to 310 of SEQ ID NO: 3 with up to ten conservative amino acid substitutions.

12. The method of claim 10, wherein the soluble form of a mammalian NgR1 comprises amino acids 26 to 344 of SEQ ID NO:4 with up to ten conservative amino acid substitutions.

13. The method of claim 10, wherein the soluble form of a mammalian NgR1 comprises amino acids 27 to 310 of SEQ ID NO:5 with up to ten conservative amino acid substitutions.

14. The method of claim 10, wherein the soluble form of a mammalian NgR1 comprises amino acids 27 to 344 of SEQ ID NO: 6 with up to ten conservative amino acid substitutions.

15. The method of claim 10, wherein said soluble form of a mammalian NgR1 comprises amino acids 26-310 of SEQ ID NO: 3 except that at least one cysteine residue is substituted with a different amino acid.

16. The method of claim 10, wherein said soluble form of a mammalian NgR1 comprises amino acids 27-310 of SEQ ID NO: 5 except that at least one cysteine residue is substituted with a different amino acid.

17. The method of claim 15, wherein amino acid C266 is substituted with a different amino acid.

18. The method of claim 15, wherein amino acid C309 is substituted with a different amino acid.

19. The method of claim 15, wherein said amino acid C266 and amino acid C309 are substituted with different amino acids.

20. The method of claim 17, wherein said different amino acid is alanine.

21. The method of claim 10, wherein the soluble form of a mammalian NgR1 further comprises a fusion moiety.

22. The method of claim 21, wherein the fusion moiety is an immunoglobulin moiety.

23. The method of claim 22, wherein the immunoglobulin moiety is an Fc moiety.

24. The method of claim 1, wherein the NgR1 antagonist comprises an antibody or antigen-binding fragment thereof that binds to a mammalian NgR1.

25. The method of claim 24, wherein the antibody is selected from the group consisting of a polyclonal antibody, a monoclonal antibody, a Fab fragment, a Fab′ fragment, a F(ab′)₂ fragment, an Fv fragment, an Fd fragment, a diabody, and a single-chain antibody.

26. The method of claim 24, wherein the antibody or antigen-binding fragment thereof binds to a polypeptide bound by a monoclonal antibody produced by a hybridoma selected from the group consisting of: HB 7E11 (ATCC® accession No. PTA-4587), HB 1H2 (ATCC® accession No. PTA-4584), HB 3G5 (ATCC® accession No. PTA-4586), HB 5B10 (ATCC® accession No. PTA-4588) and HB 2F7 (ATCC® accession No. PTA-4585).

27. The method of claim 26, wherein said monoclonal antibody is produced by the HB 7E1 1 hybridoma.

28. The method of claim 27, wherein the polypeptide comprises an amino acid sequence selected from the group consisting of:

| | |
|---|---|
| AAAFGLTLLEQLDLSDNAQLR; | (SEQ ID NO: 7) |
| LDLSDNAQLR; | (SEQ E) NO: 8) |
| LDLSDDAELR; | (SEQ ID NO: 9) |
| LDLASDNAQLR; | (SEQ ID NO: 10) |
| LDLASDD AELR; | (SEQ ID NO: 11) |
| LDALSDNAQLR; | (SEQ ID NO: 12) |
| LDALSDDAELR; | (SEQ ID NO: 13) |
| LDLSSDNAQLR; | (SEQ ID NO: 14) |
| LDLSSDEAELR; | (SEQ ID NO: 15) |
| DNAQLRWDPTT; | (SEQ ID NO: 16) |
| DNAQLR; | (SEQ ID NO: 17) |
| ADLSDNAQLRVVDPTT; | (SEQ ID NO: 18) |
| LALSDNAQLRVVDPTT; | (SEQ ID NO: 19) |
| LDLSDNAALRWDPTT; | (SEQ ID NO: 20) |
| LDLSDNAQLHVVDPTT; | (SEQ TD NO: 21) |
| and | |
| LDLSDNAQLAWDPTT. | (SEQ ID NO: 22) |

29. The method of claim 27, wherein the polypeptide consists of an amino acid sequence selected from the group consisting of:

| | |
|---|---|
| AAAFGLTLLEQLDLSDNAQLR; | (SEQ ID NO: 7) |
| LDLSDNAQLR; | (SEQ ID NO: 8) |
| LDLSDDAELR; | (SEQ E) NO: 9) |
| LDLASDNAQLR; | (SEQ E⁾ NO: 10) |
| LDLASDD AELR; | (SEQ E) NO: 11) |
| LDALSDNAQLR; | (SEQ E⁾ NO: 12) |
| LDALSDDAELR; | (SEQ E) NO: 13) |
| LDLSSDNAQLR; | (SEQ E⁾ NO: 14) |
| LDLSSDEAELR; | (SEQ TD NO: 15) |
| DNAQLRWDPTT; | (SEQ E) NO: 16) |
| DNAQLR; | (SEQ E) NO: 17) |
| ADLSDNAQLRVVDPTT; | (SEQ TD NO: 18) |
| LALSDNAQLRVVDPTT; | (SEQ E) NO: 19) |
| LDLSDNAALRWDPTT; | (SEQ TD NO: 20) |
| LDLSDNAQLHVVDPTT; | (SEQ E) NO: 21) |
| and | |
| LDLSDNAQLAWDPTT. | (SEQ TD NO: 22) |

30. The method of claim 1, wherein the therapeutically effective amount is from 0.001 mg/kg to 10 mg/kg.

31. The method of claim 30 wherein the therapeutically effective amount is from 0.01 mg/kg to 1.0 mg/kg.

32. The method of claim 31, wherein the therapeutically effective amount is from 0.05 mg/kg to 0.5 mg/kg.

33. A method of treating hearing loss or an optical neuropathy in a mammal, comprising administering to the mammal a therapeutically effective amount of an NgR1 antagonist.

34. The method of claim 1, wherein said NgR1 antagonist is 1D9 Fab.
